(19) United States
(12) Patent Application Publication
(10) Pub. No.: US 2022/0117472 A1
(43) Pub. Date: Apr. 21, 2022

(54) OPHTHALMOLOGICAL ENDOSCOPE AND USES THEREOF

(71) Applicant: WAKE FOREST UNIVERSITY HEALTH SCIENCES, Winston Salem, NC (US)

(72) Inventors: Keith WALTER, Winston Salem, NC (US); Kenneth WEINLANDER, Winston Salem, NC (US)

(21) Appl. No.: 17/431,675

(22) PCT Filed: Feb. 18, 2020
(86) PCT No.: PCT/US2020/018652

§ 371 Date: Aug. 17, 2021

Related U.S. Application Data
(60) Provisional application No. 62/806,988, filed on Feb. 18, 2019.

(51) Int. Cl. A61B 1/018; A61B 1/313; A61B 1/00; A61F 9/007

(52) U.S. Cl. CPC A61B 1/018; A61B 1/313; A61F 9/00781; A61B 1/00165; A61B 1/00126

(57) ABSTRACT

Ophthalmic endoscopes are provided that overcome deficiencies associated with current approaches to implanting ophthalmic stents and shunts for treatment of glaucoma. In various aspects, ophthalmic endoscopes are provided having an elongated body, an instrument port, a micro fiber-optic camera, and in some aspects an irrigation port, wherein the irrigation port extends at least the length of the elongated body from the proximal end to the distal end. The use of the fiber-optic camera and the angle of the irrigation port can allow the device to be operated with a single hand and to overcome the difficult positioning associated with conventional implantation procedures. Ophthalmic endoscopy systems are also provided including the endoscope and a video processor. Methods of implanting an ophthalmic implant using the ophthalmic endoscopes and endoscopy systems are also provided.

### CROSS-REFERENCE TO RELATED APPLICATIONS

This application claims priority to, and the benefit of, co-pending U.S. provisional application entitled “OPHTHALMOLOGICAL ENDOSCOPE AND USES THEREOF” having Ser. No. 62/806,988, filed Feb. 18, 2019, the contents of which are incorporated by reference in their entirety.

### TECHNICAL FIELD

The present disclosure generally relates to medical devices, and in particular to endoscopic tools useful for implanting micro-invasive glaucoma stents.

### BACKGROUND

Glaucoma is a disease of the eye in which fluid builds up in the front section of the eye causing damage to the optic nerve. This can ultimately result in vision loss and blindness. More than 120,000 of the approximate 300 million people living with glaucoma are blind and make up about 10 percent of all reported blindness. Annual costs associated with care and treatment of glaucoma exceeds $1.5 billion per year in the United States. In the early stage of the disease, it can be controlled with eye drops to reduce pressure in the anterior chamber of the eye. However, in some cases the use of eye drops is not favored, such as where compliance is an issue or when the disease has progressed to a point where drops are no longer effective. In these cases, patients often undergo stent implantation to relieve intraocular pressure. Although stenting has become popular in recent years, they can be difficult to implant.

In the last 5-6 years, Micro-Invasive Glaucoma Stents (MiGs), have become popular for surgical treatment of glaucoma. Popular stents include one made by GLAUKOS™ and called the ISTENT INJECT™, the HYDRUS™ made by IVANTIS™ and the XEN™ gel stent marketed by ALLERGAN™ in the US. These devices are used to help patients get off their glaucoma drops and are FDA approved during cataract surgery. The FDA trial data shows about 80% of patients are able to cease using at least one glaucoma drop and 50% can get off two glaucoma drops.

However, current MiGs are not without their drawbacks. In particular, they are difficult to implant. The difficulty stems primarily from issues with visualization during implant procedure. Currently, a surgeon must perform gonioscopy at the same time as implanting this device. This requires that the patient be tilted 30 degrees or more, that the microscope be tilted at least at 30 degrees, and/or that the surgeon sit or stand in an awkward position, all while placing a gonio prism gently on the eye with one hand and using the other hand with the device that implants the MiGs. Current implant procedures require performance of a gonioscopy because the MiGs are implanted at an angle into the eye at the point where the iris meets the cornea, which is not directly visible with the operating microscope. The multiple instruments that must be handled simultaneously by the surgeon coupled with the awkward positioning of both patient and surgeon increases the risk of the procedure to the patient. As such, there is a need for improved for devices and techniques for surgical treatment of glaucoma.

### SUMMARY

Considering the problems associated with current glaucoma micro-stenting procedures, described herein are devices and methods that overcome one or more of the aforementioned deficiencies. In particular, provided are ophthalmic endoscopes configured for direct visualization of the eye during implantation of an ophthalmic implant (such as a MiGs) and other devices into the anterior region of the eye. Also described herein are methods of implanting an ophthalmic implant (such as a MiGs) into an eye using direct endoscopic visualization of the eye during the procedure. Also provided are ophthalmic endoscopes that, while providing for direct visualization of the eye during implantation of the ophthalmic implant, further provided for appropriate drainage through one or more drainage ports. This is difficult, if not impossible, to simultaneously accomplish with current endoscopic tools because of the angles involved. Methods of using these devices are also provided.

In some aspects, a method is provided for implanting an ophthalmic implant into an eye of a subject in need thereof, the method including the steps of making an incision into the anterior region of the eye; inserting a distal end of an ophthalmic endoscope into the incision, wherein the ophthalmic endoscope is any ophthalmic endoscope described herein; and inserting a distal end of an applicator tool to implant an ophthalmic implant.

In some aspects, the ophthalmic endoscope includes an elongated body, wherein the elongated body has a distal end and a proximal end, wherein the proximal end and the distal end each comprise a cross-sectional width, wherein the cross-sectional width of the distal end is smaller than the cross-sectional width of the proximal end. The instrument and/or ophthalmic implant can thereby be passed through the instrument port of the endoscope for implanting an ophthalmic implant into a region of the eye.

The devices and/or methods described herein can have the advantage of reducing the complexity of the MiGs implant procedure, thereby reducing the risk to the patient. Other devices, methods, features, and advantages of the present disclosure will be or become apparent to one having ordinary skill in the art upon examination of the following drawings, detailed description, and examples. It is intended that all such additional devices, methods, features, and advantages be included within this description, and be within the scope of the present disclosure.

### BRIEF DESCRIPTION OF THE DRAWINGS

Further aspects of the present disclosure will be readily appreciated upon review of the detailed description of its various embodiments, described below, when taken in conjunction with the accompanying drawings.

FIG. 1 shows a top/side perspective view of a first exemplary ophthalmic endoscope according to various aspects of the disclosure.

FIG. 2 shows a cross-sectional view of a distal end of the first exemplary ophthalmic endoscope of FIG. 1.

FIG. 3 shows a cross-sectional view of a proximal end of the first exemplary ophthalmic endoscope of FIG. 1.

### DETAILED DESCRIPTION

In various aspects, ophthalmic endoscopes and methods of use thereof are provided that overcome one or more of the aforementioned problems. In particular aspects, ophthalmic endoscopes are provided that allow for improved visibility of the anterior region of the eye during the implantation of a Micro-Invasive Glaucoma Stents or similar ophthalmic implant, even without the use of a gonioscopic lens. In particular aspects, ophthalmic endoscopes are provided that can be operated with a single hand during the implantation procedure, thereby sampling operation and reducing complications. In particular aspects, ophthalmic endoscopes are provided with an irrigation port that is angled away from the instrument port to ensure proper irrigation/drainage during use. Methods of using the ophthalmic endoscope are also provided.

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting. The skilled artisan will recognize many variants and adaptations of the embodiments described herein. These variants and adaptations are intended to be included in the teachings of this disclosure.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents. Any lexicographical definition in the publications and patents cited that is not also expressly repeated in the instant specification should not be treated as such and should not be read as defining any terms appearing in the accompanying claims. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

Although any methods and materials similar or equivalent to those described herein can also be used in the practice or testing of the present disclosure, the preferred methods and materials are now described. Functions or constructions well-known in the art may not be described in detail for brevity and/or clarity. Embodiments of the present disclosure will employ, unless otherwise indicated, surgical techniques involving the eye such as would typically be performed by an ophthalmologist or similarly licensed medical professional. Such techniques are within the skill of the art and are explained fully in the literature.

It should be noted that ratios, concentrations, amounts, and other numerical data can be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to about 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.2%, 3.3%, and 4.4%) within the indicated range. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In some embodiments, the term “about” can include traditional rounding according to significant figures of the numerical value. In addition, the phrase “about ‘x’ to ‘y’”, where ‘x’ and ‘y’ are numerical values, includes “about ‘x’ to about ‘y’”.

In some instances, units may be used herein that are non-metric or non-SI units. Such units may be, for instance, in U.S. Customary Measures, e.g., as set forth by the National Institute of Standards and Technology, Department of Commerce, United States of America in publications such as NIST HB 44, NIST HB 133, NIST SP 811, NIST SP 1038, NBS Miscellaneous Publication 214, and the like. The units in U.S. Customary Measures are understood to include equivalent dimensions in metric and other units (e.g., a dimension disclosed as “1 inch” is intended to mean an equivalent dimension of “2.5 cm”; a unit disclosed as “1 pcf” is intended to mean an equivalent dimension of 0.157 kN/m³; or a unit disclosed 100° F. is intended to mean an equivalent dimension of 37.8° C.; and the like) as understood by a person of ordinary skill in the art.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

The articles “a” and “an,” as used herein, mean one or more when applied to any feature in embodiments of the present invention described in the specification and claims. The use of “a” and “an” does not limit the meaning to a single feature unless such a limit is specifically stated. The article “the” preceding singular or plural nouns or noun phrases denotes a particular specified feature or particular specified features and may have a singular or plural connotation depending upon the context in which it is used.

Terms of orientation such as “left,” “right,” “top,” “bottom,” “upper,” “lower,” “front,” “rear,” and “end” are used herein to simplify the description of the context of the illustrated aspects. Likewise, terms of sequence, such as “first” and “second,” are used to simplify the description of the illustrated aspects. Because other orientations and sequences are possible, however, the claims should not be limited to the illustrated orientations or sequences. Those skilled in the art will appreciate, upon reading this disclosure, that other orientations of the various components described above are possible.

The following description will include references to distal and proximal ends of various components and right and left sides of various components. The terms “distal” and “proximal” are to be given their ordinary and customary meaning to a person of ordinary skill in the art (and are not to be limited to a special or customized meaning), and refer without limitation to opposite regions or ends of a particular structure. In some aspects, the term “distal” is used to refer to a region or end farther away from a person using the systems and devices described herein or performing the methods described herein and the term “proximal” is used to refer to a region or end closer to the person using the systems and devices described herein or performing the methods described herein; however, the meanings of the terms can be swapped as will be understood from context.

The term “glaucoma,” colloquially also termed “Gruener Star”, is used herein to refer to a group of ophthalmic diseases characterized by a temporarily or permanently increased intraocular pressure which can obstruct the blood supply of the optic nerve. The term glaucoma includes, for example, primary open-angle glaucoma (“POAG”), progressive low-tension glaucoma, exfoliation and open-angle glaucoma (“OAG”), amylodosis and open-angle glaucoma, pigment dispersion and pigmentary glaucoma, angle-closure glaucoma, combined open-angle and angle-closure glaucoma, malignant glaucoma, angle-closure glaucoma after scleral buckling operations for separated retina, angle-closure glaucoma due to a multiple cyst of iris and ciliary body, angle-closure glaucoma secondary to occlusion of the central retinal vein, angle-closure glaucoma secondary to bilateral transitory myopia, glaucoma from perforating injuries, glaucoma from contusion of the eye, hemolytic or ghost-cell glaucoma, glaucoma associated with congenital and spontaneous dislocations of the lens, lens-induced glaucoma, glaucoma in aphasia, glaucoma due to intraocular inflammation, neovascular glaucoma, glaucoma associated with extra ocular venous congestion, essential atrophy of the iris with glaucoma, corticosteroid glaucoma, glaucoma after penetrating keratoplasty and characteristically unilateral glaucomas, and other primary and secondary glaucomas, as well as glaucoma related diseases and disorders, see e.g. Chandler et al. (Glaucoma, 3d Ed., Lea and Febliger, Philadelphia (1986)). In almost all cases, the IOP found in glaucoma results from an increase in aqueous outflow resistance (see, Vaughan, D. et al., In: General Ophthamology, Appleton & Lange, Norwalk, Conn., pp. 213-230 (1992)). A glaucoma related disease or disorder includes any disease or condition that has or displays a symptom of glaucoma, e.g. elevated intraocular pressure resulting from water leakage resistance. In ordinary terminology, glaucoma is called “primary” if the pathogenic defect is believed to occur primarily within the tissue itself and without an obvious outside causal mechanism which can be defined for “secondary” glaucomas (e.g., see McGraw-Hill Encyclopedia of Science and Technology, 6th Ed., Vol. 8, p. 131 (McGraw-Hill 1987).

As used interchangeably herein, the terms “ophthalmic implant” and “implant” refer to ocular implants or drug delivery devices that can be implanted into any number of locations in the eye, including those that may release a controlled amount of a bioactive agent or therapeutic immediately or over time. The term implant includes micro-invasive glaucoma stents and shunts, which are used interchangeably herein to refer to a class of implants designed for non-invasive or minimally-invasive implantation in the eye and that have one or more hollow microfistula tubes or flow paths through which aqueous humour can pass, e.g. to pass from the anterior chamber of the eye into a targeted tissue located there behind.

### Ophthalmic Endoscopes and Methods of Use Thereof

In the last 5-6 years, Micro-Invasive Glaucoma Stents (MiGs), have become popular in ophthalmology, particularly for surgical treatment of glaucoma. The most popular one is made by Glaukos and is called the iStent inject, which is Glaukos's newest FDA approved version. Other products made by others is the Hydrus and XEN implant. Recently the Cypass was recalled due to some design flaws but will be back on the market. These devices are used to help patients get off their glaucoma drops and are FDA approved during cataract surgery. The FDA trial data shows about 80% of patient can get off one glaucoma drop and 50% can get off two glaucoma drops.

However, current MiGs are not without their drawbacks. In particular, they are difficult to implant. The difficulty stems primarily from issues with visualization during implant procedure. Currently, a surgeon must perform gonioscopy at the same time as implanting this device. This requires that the patient be tilted 30 degrees or more, that the microscope be tilted at least at 30 degrees, and/or that the surgeon sit or stand in an awkward position, all while placing a gonio prism gently on the eye with one hand and using the other hand with the device that implants the MiGs. Current implant procedures require performance of a gonioscopy because the MiGs are implanted at an angle into the eye at the point where the iris meets the cornea, which is not directly visible with the operating microscope. The multiple instruments that must be handled simultaneously by the surgeon coupled with the awkward positioning of both patient and surgeon increases the risk of the procedure to the patient. As such, there is a need for improved for devices and techniques for surgical treatment of glaucoma.

With that said, described herein are devices configured for direct visualization of the eye during implantation of an ophthalmic implant (such as a MiGs) and other devices into the anterior region of the eye. Also described herein are methods of implanting an ophthalmic implant (such as a MiGs) into an eye using direct endoscopic visualization of the eye during the procedure. The devices and/or methods described herein can have the advantage of reducing the complexity of the MiGs implant procedure, thereby reducing the risk to the patient. Other compositions, compounds, methods, features, and advantages of the present disclosure will be or become apparent to one having ordinary skill in the art upon examination of the following drawings, detailed description, and examples. It is intended that all such additional compositions, compounds, methods, features, and advantages be included within this description, and be within the scope of the present disclosure.

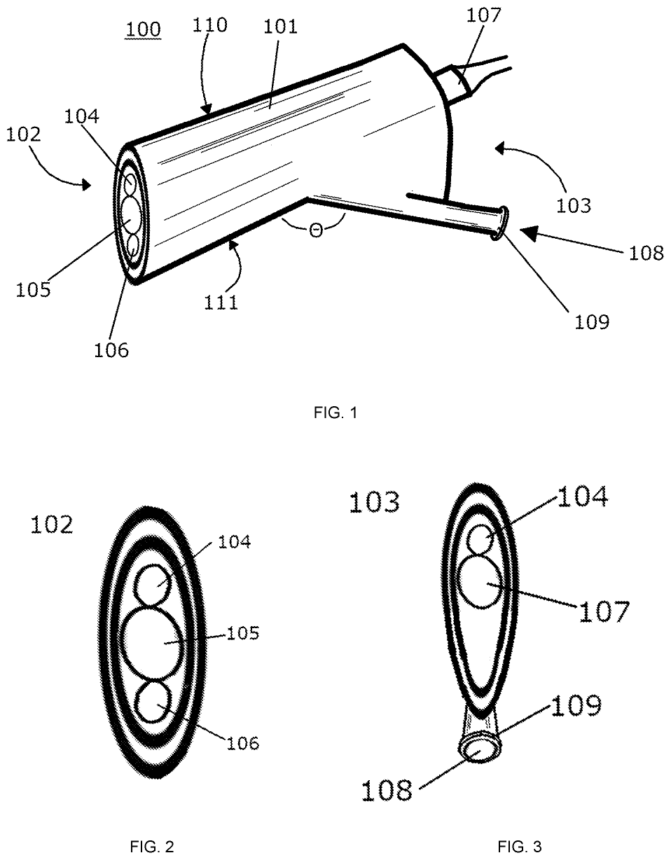

As shown in FIGS. 1-3, an exemplary ophthalmic endoscope 100 can have an elongated body 101. The elongated body can have a distal end 102 and a proximal end 103. The ophthalmic endoscope 100 can have an instrument port 104. The instrument port 104 can form a cannula that can extend the length of the elongated body 101 from the proximal end 103 to the distal end 102, can be configured to receive an instrument and/or ophthalmic implant (not pictured), and can allow passage of the instrument and/or ophthalmic implant through the elongated body 101 from the proximal end 103 to the distal end 102.

The ophthalmic endoscope 100 can further include a micro fiber optic camera that can have an optical fiber 107 and a lens 105, where the optical fiber 107 can be optically coupled to the lens 105, where the lens 105 is at the distal end 102 of the elongated body 101, where the optical fiber 107 can extend at least the length of the elongated body 101, and where the optical fiber 107 can be configured to optically couple to a video processor (not pictured). The micro-fiber optic camera can have a field of vision that can range from about 100° to about 140°.

The video processor can be optically coupled to the optical fiber of the ophthalmic endoscope and can be configured to process an optical signal received from the micro optical fiber camera into a video image signal. The monitor can be coupled to the video processor and configured to receive the video image signal and display a video image produced from the video image signal. It will be appreciated that monitor can be wirelessly coupled or can be coupled via a suitable video cable to the video processor. In some embodiments, the monitor includes the video processor. During use the monitor can display an image from the field of view of the camera to the surgeon. In this way the ophthalmic endoscopy can be used by the surgeon to view the internal regions of the eye during an eye procedure, such as the one described herein.

The ophthalmic endoscope 100 can further have an irrigation port 106. The irrigation port 1-6 can extend at least the length of the elongated body 101 from the proximal end 103 to the distal end 102. The proximal end of the irrigation port 108 can have a locking mechanism 109 that can be configured to receive a male end or female end of a Leuer lock.

In some aspects, the ophthalmic endoscope is substantially straight along its length. This can occur when the cross-sectional width and length of the distal and proximal ends are the same. In other aspects, for example as depicted in the exemplary ophthalmic endoscope 100 one or more sides can be angled such that a point of deflection can occur at any point along the length of one or more sides to form the angle. In some aspects, such as that shown most clearly in FIG. 1, one side 110 of the elongated body 101 can be substantially straight along the entire length of the elongated body 101 and ban opposing side 111 of the elongated body 101 can be angled by an angle (Θ) at a point along the length of the elongated body 101. The angle (Θ) can range from about 120 degrees to about 170 degrees. This can allow for direct visualization when performing an implantation procedure for a glaucoma stent or shunt. This can also be advantageous when utilizing a microfiber optic camber with a smaller field of vision.

The elongated body can have a distal end 102 and a proximal end 103 each having a cross-sectional width, where cross-sectional width of the distal end is smaller than the cross-sectional width of the proximal end. The cross-sectional width of the distal end can range from about 1 mm to about 3 mm. In some embodiments, the cross-sectional width of the distal end is smaller than the cross-sectional width of the proximal end. In some embodiments, the cross-sectional width of the distal end is the same as the cross-sectional width of the proximal end. In some embodiments the cross-sectional width of the distal end can be about 1.2 mm. The distal end and the proximal end can each have a cross-sectional length. In some embodiments, the cross-sectional length of the distal end can be smaller than the cross-sectional length of the proximal end. In some embodiments, the cross-sectional length of the distal end can be same as the cross-sectional length of the proximal end. In some embodiments, the cross-sectional length of the distal end can range from about 1 to about 6 mm. In some embodiments, the cross-sectional length of the distal end is about 2.4 mm. In some embodiments, the cross-sectional shape of the distal end and/or proximal end can be oval. In some embodiments, the distal end and/or the proximal end(s) can have other regular or irregular cross-sectional shapes. The ophthalmic implant can be a MiGs. Exemplary MiGs include, but are not limited to, the iStent implant, Hydrus implant, Cypass implant, and XEN implant.

Described herein are aspects of a method of implanting an ophthalmic implant (such as a Micro-Invasive Glaucoma Stent (MiGs)) into an eye of a subject in need thereof that can include the steps of making an incision into the anterior region of the eye; inserting an ophthalmic endoscope into the incision, passing an instrument and/or applicator tool having an ophthalmic implant through the instrument port of the endoscope; and implanting an ophthalmic implant into a region of the eye.

The method can be performed as a stand-alone implant placement procedure or can be combined with another procedure such as cataract removal surgery. The incision can be a primary incision or a secondary incision (e.g. one performed for paracentesis). The incision can be a minimally invasive incision. The incision through which the distal region of the ophthalmic endoscope can be inserted can be about 1 mm to about 3 mm. In some embodiments, the MiGs can be implanted into Schlem's canal.

The method can include performing a cataract procedure through a temporal clear corneal incision. For some patients with primary angle closure and primary angle closure glaucoma, it may make sense to remove the cataract as part of a treatment plan for glaucoma. Additionally, evidence suggests that cataract removal by itself in open-angle glaucoma can also lower eye pressure by a few points. The procedures can include performing an anterior capsulorhexis. At this point, the area is typically inflated with viscoelastic and the patient and microscope are tilted so that the implantation site can be viewed through a goniscopic lens. Using the endoscopes described herein can overcome this difficulty, allowing the ophthalmologist to directly access and view the implantation site. The implant can be placed using an applicator tool passed through the instrument port, where the implant at a distal end of the applicator is viewed by aide of the optic camera and guided into the implantation site by the operator. Once in place, the applicator tool can be released and (if needed) the implant can be further positioned or tapped into place. The viscoelastic can then be removed and the wound hydrated.

1. A method of implanting an ophthalmic implant into an eye of a subject in need thereof, the method comprising:
making an incision into an anterior region of the eye;
inserting an ophthalmic endoscope into the incision, wherein the ophthalmic endoscope comprises:
(i) an elongated body, wherein the elongated body comprises a distal end and a proximal end, wherein the proximal end and the distal end each comprise a cross-sectional width, wherein the cross-sectional width of the distal end is smaller than the cross-sectional width of the proximal end, wherein a first side of the elongated body is substantially straight along the entire length of the elongated body and wherein a second side opposite the first side of the elongated body is angled at a point along the length of the elongated body, and wherein the angle formed in the second side of the elongated body ranges from about 120 degrees to 170 degrees;
(ii) an instrument port, wherein the instrument port forms a cannula extending a length of the elongated body from the proximal end to the distal end, wherein the instrument port is configured to receive an instrument and/or applicator tool for an ophthalmic implant, and wherein the instrument port is further configured to allow passage of the instrument and/or applicator tool containing the ophthalmic implant through the elongated body from the proximal end to the distal end; and
(iii) a micro fiber optic camera comprising an optical fiber and a lens, wherein the optical fiber is optically coupled to the lens, wherein the lens is at the distal end of the elongated body, and wherein the optical fiber extends at least the length of the elongated body and is configured to optically couple to a video processor
passing an instrument and/or ophthalmic implant through the instrument port of the endoscope; and
implanting the ophthalmic implant into a region of the eye.

2. The method of claim 1, wherein the ophthalmic implant is a micro-invasive glaucoma stent or shunt.

3. The method claim 1, wherein the incision is a minimally invasive incision.

4. The method of claim 1, wherein the incision is about 3 mm or less.

5. The method of claim 1, wherein the region in which the ophthalmic implant is implanted is the Schlem's canal.

6. The method of claim 1, wherein the cross-sectional width of the distal end is about 1.2 mm.

7. The method of claim 1, wherein the distal end and the proximal end each have a cross-sectional length and wherein the cross-sectional length of the distal end is smaller than the cross-sectional length of the proximal end.

8. The method of claim 7, wherein the cross-sectional length of the distal end is about 2.4 mm.

9. (canceled)

10. (canceled)

11. The method of claim 1, wherein the second side comprises a drainage port extending from the distal end to the proximal end.

12. The method of claim 1, wherein the distal end, the proximal end, or both the distal end and the proximal end are substantially oval.

13. An ophthalmic endoscope comprising:
an elongated body, wherein the elongated body comprises a distal end and a proximal end, wherein the distal end and the proximal end each have a cross-sectional width, and wherein the cross-sectional width of the distal end is smaller than the cross-sectional width of the proximal end, wherein a first side of the elongated body is substantially straight along the entire length of the elongated body and wherein a second side opposite the first side of the elongated body is angled at a point along the length of the elongated body, and wherein the angle formed in the second side of the elongated body ranges from about 120 degrees to 170 degrees;
an instrument port, wherein the instrument port forms a cannula extending the length of the elongated body from the proximal end to the distal end, wherein the instrument port is configured to receive an instrument and/or applicator tool for an ophthalmic implant, and wherein the instrument port is further configured to allow passage of the instrument and/or applicator tool with the ophthalmic implant through the elongated body from the proximal end to the distal end; and
a micro fiber optic camera comprising an optical fiber and a lens, wherein the camera is optically coupled to the lens, wherein the lens is coupled to the distal end of the elongated body, and wherein the optical fiber extends at least the length of the elongated body and is configured to optically couple to a video processor.

14. The ophthalmic endoscope of claim 13, further comprising an irrigation port, wherein the irrigation port extends at least the length of the elongated body from the proximal end to the distal end, wherein the proximal end of the irrigation port can be configured to receive a male end or female end of a Leuer lock.

15. The ophthalmic endoscope of claim 14, wherein the ophthalmic implant is a micro-invasive glaucoma stent or shunt.

16. The ophthalmic endoscope of claim 13, wherein the cross-sectional width of the distal end is about 1.2 mm.

17. The ophthalmic endoscope of claim 13, wherein the distal end and the proximal end each have a cross-sectional length and wherein the cross-sectional length of the distal end is small then than the cross-sectional length of the proximal end.

18. The ophthalmic endoscope of claim 17, wherein the cross-sectional length of the distal end is about 2.4 mm.

19. (canceled)

20. (canceled)

21. The ophthalmic endoscope of claim 13, wherein the distal end, the proximal end, or both the distal end and the proximal end are substantially oval.

22. An ophthalmic endoscopy system comprising:
an ophthalmic endoscope as in claim 1;
a video processor, wherein the video processor is optically coupled to the optical fiber of the ophthalmic endoscope and is configured to process an optical signal received from the micro optical fiber camera into a video image; and
a monitor, wherein the monitor is coupled to the video processor and configured to receive and display the video image.

23. The ophthalmic endoscopy system of claim 22, wherein the video processor is wirelessly coupled to the monitor.

24. The ophthalmic endoscopy system of claim 22, wherein the video processor is coupled to the monitor via a suitable video cable.
